(19) United States
(12) Patent Application Publication
(10) Pub. No.: US 2021/0361146 A1
(43) Pub. Date: Nov. 25, 2021

(54) ENDOSCOPE, AND INSERTION PORTION OF ENDOSCOPE

(71) Applicant: OLYMPUS CORPORATION, Tokyo (JP)

(72) Inventors: Eita IYOSHI, Hachioji-shi, Tokyo (JP); Yoshimi KONNO, Akiruno-shi, Tokyo (JP); Naoto KIKUCHI, Hino-shi, Tokyo (JP)

(73) Assignee: OLYMPUS CORPORATION, Tokyo (JP)

(21) Appl. No.: 17/398,397

(22) Filed: Aug. 10, 2021

Related U.S. Application Data
(63) Continuation of application No. PCT/JP2019/005877, filed on Feb. 18, 2019.

(51) Int. Cl. A61B 1/00; A61B 1/05; C09J 11/04

(52) U.S. Cl. CPC A61B 1/00135; A61B 1/05; C08K 2003/265; C09J 11/04; A61B 1/0011

(57) ABSTRACT

An endoscope includes: an image pickup unit having an incidence face; a distal end body including a front face in which an insertion hole is formed, the incidence face of the image pickup unit inserted into the insertion hole protruding from the front face; a distal end cover that covers a periphery of the insertion hole of the distal end body and has a distal end face including an opening from which the incidence face is protruded; a first adhesive that bonds the distal end cover to the image pickup unit and contains first calcium carbonate particles; and a second adhesive that covers a step at a boundary between the distal end face of the distal end cover and the incidence face of the image pickup unit and contains first silicon oxide particles.

### CROSS REFERENCE TO RELATED APPLICATION

This application is a continuation application of PCT/JP2019/005877 filed on Feb. 18, 2019, the entire contents of which are incorporated herein by this reference.

### BACKGROUND OF THE INVENTION

### 1. Field of the Invention

The present invention relates to an endoscope in which an image pickup unit is arranged in a distal end portion of an insertion portion, and the insertion portion of the endoscope.

### 2. Description of the Related Art

An endoscope is widely used that is configured to observe an inner part of a body of a subject by using an image pickup unit arranged in a distal end portion, by an elongated insertion portion being inserted into the inner part of the body, which cannot be observed from the outside, and to perform treatment by using a treatment instrument protruded from an opening of a channel.

The image pickup unit is fixed in a state of being inserted into an insertion hole of a distal end body which is arranged in the distal end portion. The distal end cover is bonded to the distal end body using an adhesive. An incidence face of the image pickup unit protrudes from a front face of the distal end cover.

International Publication No. 2011/126018 discloses a technology of fixing an image pickup unit by using a curable resin containing spherical silica, and eliminating a step on the surface.

### SUMMARY OF THE INVENTION

An endoscope according to an embodiment includes: an image pickup unit having an incidence face; a distal end body including a front face in which an insertion hole is formed, the incidence face of the image pickup unit inserted into the insertion hole protruding from the front face; a distal end cover that covers a periphery of the insertion hole of the distal end body and has a distal end face including an opening from which the incidence face is protruded; a first adhesive that bonds the distal end cover to the image pickup unit and contains first calcium carbonate particles; and a second adhesive that covers a step at a boundary between the distal end face of the distal end cover and the incidence face of the image pickup unit and contains first silicon oxide particles.

An insertion portion of an endoscope of another embodiment includes: an image pickup unit having an incidence face; a distal end body including a front face in which an insertion hole is formed, the incidence face of the image pickup unit inserted into the insertion hole protruding from the front face; a distal end cover that covers a periphery of the insertion hole of the distal end body and has a distal end face including an opening from which the incidence face is protruded; a first adhesive that bonds the distal end cover to the image pickup unit and contains first calcium carbonate particles; and a second adhesive that covers a step at a boundary between the distal end face of the distal end cover and the incidence face of the image pickup unit and contains first silicon oxide particles.

### BRIEF DESCRIPTION OF THE DRAWINGS

FIG. 1 is an external view of an endoscope of an embodiment;

FIG. 2 is a plan view of a distal end portion of the endoscope of the embodiment;

FIG. 3 is a cross-sectional view taken along the line III-III in FIG. 2; and

FIG. 4 is an enlarged view of a portion indicated by (IV) in FIG. 3.

### DETAILED DESCRIPTION OF THE PREFERRED EMBODIMENTS

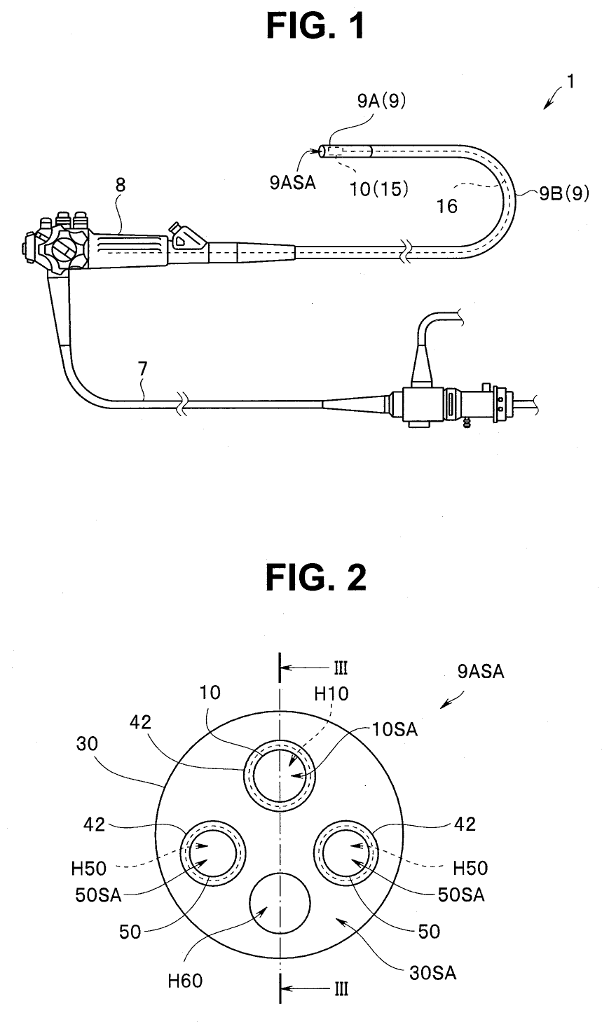

As shown in FIG. 1, an endoscope 1 of an embodiment includes: an insertion portion 9 including a distal end portion 9A and an elongated flexible portion 9B; an operation section 8 arranged in the insertion portion 9; and a universal cord 7 extending from the operation section 8.

Note that in the following description, the drawings based on each embodiment are schematic. Relationships between thickness and width of each portion, ratios of thickness of each portion and the like are different from actual relationships and ratios. Also among the mutual drawings, portions are contained in which relationships and ratios between mutual dimensions are different. Some components are not illustrated or denoted by reference numerals in the drawings.

An image pickup signal that has been outputted from an image pickup portion 15 of the image pickup unit 10 arranged in the distal end portion 9A is transmitted to a processor (not illustrated) via a signal cable 16 that allows insertion of the flexible portion 9B, the operation section 8 and the universal cord 7.

Note that though the endoscope 1 is a flexible endoscope having the flexible portion 9B, the endoscope of the embodiment may be a rigid endoscope. The endoscope 1 may be used for medical or industrial application.

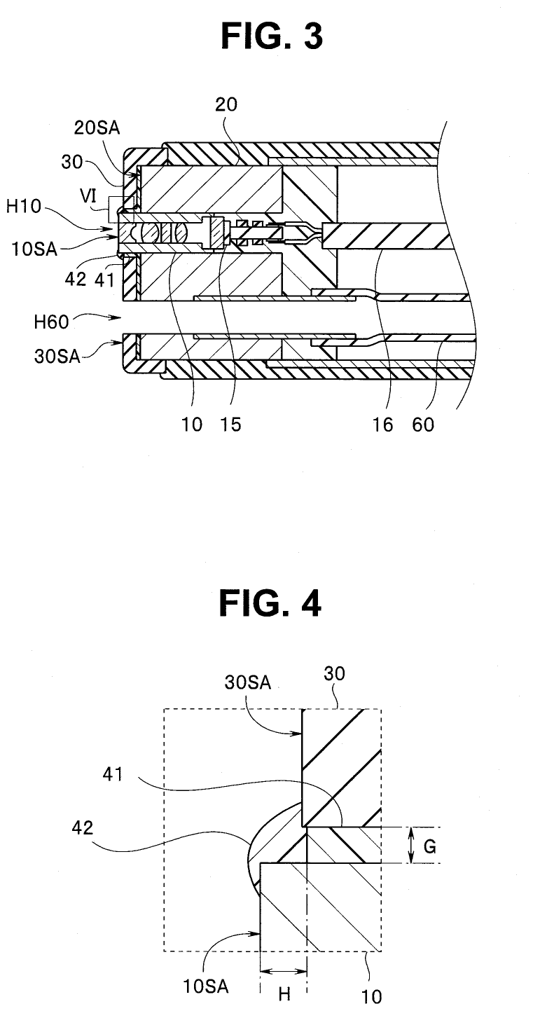

As shown in FIG. 2 and FIG. 3, a distal end body 20 is arranged on the distal end portion 9A of the insertion portion 9. The distal end body 20 is, for example, a substantially cylindrical housing made of stainless steel.

A plurality of insertion holes H10, H50 and H60 are provided on a front face 20SA of the distal end body 20. The image pickup unit 10 is inserted in and fixed to an insertion hole H10. Illumination units 50 are inserted in and fixed to two insertion holes H50, respectively. An insertion hole H60 constitutes an opening of a channel 60 through which a treatment instrument or the like is inserted.

In the distal end body 20, at least a part of peripheries and side faces of the insertion holes H10, H50 and H60 is covered with a distal end cover 30 made from polysulfone, for example.

Note that the resin constituting the distal end cover 30 is not limited to polysulfone, but may be any resin having biocompatibility, chemical resistance and non-conductivity.

The incidence face 10SA of the image pickup unit 10 and the emission face 50SA of the illumination unit 50 protrude from the distal end face 30SA of the distal end cover 30.

In other words, in the distal end cover 30 that is exposed on an end face 9ASA of the distal end portion 9A, there are a plurality of holes communicating with the insertion holes H10, H50 and H60, respectively.

The distal end cover 30 is bonded to the image pickup unit 10 and the illumination unit 50 using a first adhesive 41.

As shown in FIG. 4, a gap G between the distal end cover 30 and the image pickup unit 10, which is filled with the first adhesive 41, is 10 μm to 100 μm, for example. A metallic frame for an optical system of the image pickup unit 10 is bonded to the distal end cover 30, but, for example, the foremost lens of the optical system may be bonded to the distal end cover 30.

Note that the image pickup unit 10 and the illumination unit 50 may be bonded to the distal end body 20 using the first adhesive 41.

The incidence face 10SA of the image pickup unit 10 protrudes ahead of the distal end face 30SA of the distal end cover 30. A step H between the incidence face 10SA and the distal end face 30SA is 10 μm to 150 μm, for example.

As shown in FIG. 2, in order to eliminate the step between the incidence face 10SA and the distal end face 30SA, a second adhesive 42 is arranged along an outer periphery of the incidence face 10SA, in a ring shape. Accordingly, the endoscope 1 is excellent in drainage; and because water droplets do not adhere to the incidence face 10SA, is excellent in operability.

A resin of each of the first adhesive 41 and the second adhesive 42 is, for example, a thermosetting resin containing a mixture of an epoxy resin and an acrylic rubber. A curable resin shrinks at the time of curing, and accordingly, it is preferable that the curable resin contain a filler (particles) which does not shrink, in order to prevent detachment at the time of curing.

The inventor has found that a resin containing calcium carbonate particles has a smaller thixotropic index T than a resin containing silicon oxide particles. Furthermore, the inventor has found that a resin which contains non-spherical particles, in other words, cubic, spindle, rod-shaped or amorphous calcium carbonate particles, has a smaller thixotropic index T than a resin which contains spherical calcium carbonate particles.

The thixotropic index T is a ratio between viscosities at different shear rates. In the present invention, the viscosity was measured by using a B-type rotational viscometer (Brookfield-type viscometer), according to JIS Z8803. A value of viscosity ηB which has been measured at a rotation speed of 0.5 rpm (room temperature) relative to viscosity ηA which has been measured at a rotation speed of 100 rpm (room temperature) is determined as the thixotropic index T.

T=ηB/ηA

Note that the viscosity which has been measured at the rotation speed higher than 100 rpm did not differ significantly from the viscosity ηA which has been measured at the rotation speed of 100 rpm, and that the viscosity which has been measured at a rotation speed lower than 0.5 rpm did not differ significantly from the viscosity ηB which has been measured at a rotation speed of 0.5 rpm.

In other words, the viscosity at a rotation speed corresponding to a sufficiently high shear rate is the viscosity ηA, and the viscosity at a rotation speed corresponding to a sufficiently low shear rate is the viscosity ηB.

The first adhesive 41 contains 10% by mass or more and 60% by mass or less of first calcium carbonate particles. On the other hand, the second adhesive 42 contains 10% by mass or more and 60% by mass or less of first silicon oxide particles. The first adhesive 41 and the second adhesive 42 containing the respective particles have a small cure shrinkage, and do not become detached at the time of curing.

The first calcium carbonate particles are amorphous and the first silicon oxide particles are spherical. In each case, a particle size is about 1 μm to 5 μm. The particle size of the amorphous particles is an average value of a major axis length and a minor axis length.

Here, coating workability is considered, and then a preferable viscosity of the adhesive at the time of use (at the time of coating) is substantially the same for both of the first adhesive 41 and the second adhesive 42.

The first adhesive 41 needs to fill a narrow space between the image pickup unit 10 and the distal end cover 30. Accordingly, the first adhesive 41 containing the calcium carbonate particles is used, which does not increase the viscosity even after having been coated, that is, which has a small thixotropic index T.

On the other hand, in order to eliminate the step between the incidence face 10SA and the distal end face 30SA, the second adhesive 42 containing the silicon oxide particles is used, which increases the viscosity after having been coated, and has a high thixotropic index T.

Note that the first adhesive 41 may contain second silicon oxide particles in an amount smaller than an amount of the first calcium carbonate particles. On the contrary, the second adhesive 42 may contain a smaller amount of the second calcium carbonate particles than an amount of the first silicon oxide particles. It is easier for an adhesive containing different types of particles to obtain a desired viscosity and a desired thixotropic property, due to the combination, than for an adhesive containing one type of particle.

For example, the first adhesive 41 contains 30% by mass of non-spherical calcium carbonate particles (particle size of 5 μm), and further contains 1% by mass of spherical silicon oxide particles (particle size of 3 μm). The second adhesive 42 contains 20% by mass of spherical silicon oxide particles (particle size of 3 μm) and does not contain the calcium carbonate particles.

The endoscope 1 produced by use of the above first adhesive 41 and the above second adhesive 42 had excellent coating workability, the production was easy, the adhesion reliability was high, and the step between the incidence face 10SA and the distal end face 30SA was eliminated. Accordingly, the endoscope 1 is excellent in the operability.

A viscosity ηA31 of the first adhesive 41 was 12.0 Pa·s, which was measured at the rotation speed of 100 rpm. On the other hand, a viscosity ηA32 of the second adhesive 42 was 12.7 Pa·s, which was measured under the same conditions.

When the viscosity ηA31 at the rotation speed of 100 rpm which is considered as a condition corresponding to a shear rate at the time of coating is substantially the same as the viscosity ηA32, for example, when the viscosity ηA31 is 50% or more and 200% or less of the viscosity ηA32, the coating workability is excellent.

On the other hand, the viscosity ηB31 of the first adhesive 41 was 13.2 Pa·s, which was measured at a rotation speed of 0.5 rpm that was considered to correspond to a shear rate at the time when having been left after having been coated. On the other hand, the viscosity ηB32 of the second adhesive 42 was 19.1 Pa·s, which was measured under the same conditions.

The thixotropic index T41 of the first adhesive 41 is 1.10. The thixotropic index T42 of the second adhesive 42 is 1.50. The thixotropic index of the first adhesive 41 is smaller than the thixotropic index of the second adhesive 42.

A ratio of thixotropic index T42 to thixotropic index T41 (T42/T41) is 1.36.

It is preferable for the thixotropic index T41 to be 1.0 or larger and 1.9 or smaller, and is more preferable to be 1.5 or smaller. It is preferable for the thixotropic index T42 to be 1.1 or larger and 2.0 or smaller, and is more preferable to be 1.5 or larger. Furthermore, it is preferable for (T42/T41) to be 1.10 or larger, and is more preferable to be 1.25 or larger.

When the viscosity of the first adhesive 41 and the viscosity of the second adhesive 42 are within the above ranges, the gap can be reliably filled and the step can be eliminated.

Note that it is preferable for the first adhesive 41 to contain 10% by mass or more and 40% by mass or less of non-spherical calcium carbonate particles, and to contain 0% by mass or more and 20% by mass or less of spherical silicon oxide particles. It is further preferable for the first adhesive 41 to contain 10% by mass or more and 30% by mass or less of non-spherical calcium carbonate particles, and 0% by mass or more and 10% by mass or less of spherical silicon oxide particles. However, in the first adhesive 41, the content rate of the silicon oxide particles is smaller than that of the calcium carbonate particles.

It is preferable for the second adhesive 42 to contain 10% by mass or more and 50% by mass or less of spherical silicon oxide particles, and to contain 0% by mass or more and 20% by mass or less of non-spherical calcium carbonate particles. It is further preferable for the second adhesive 42 to contain 10% by mass or more and 40% by mass or less of silicon oxide particles, and to contain 0% by mass or more and 10% by mass or less of calcium carbonate particles. However, in the second adhesive 42, the content rate of the calcium carbonate particles is smaller than that of the silicon oxide particles.

When the content rates in the first adhesive 41 and the second adhesive 42 are within the above ranges, the endoscope 1 has excellent coating workability, the production is easy, the adhesion reliability is high, and the step between the incidence face 10SA and the distal end face 30SA is eliminated. Accordingly, the endoscope 1 is excellent in the operability.

Note that the second silicon oxide particles contained in the first adhesive 41 may be different from the first silicon oxide particles contained in the second adhesive 42, and that the second calcium carbonate particles contained in the second adhesive 42 may be different from the first calcium carbonate particles contained in the first adhesive 41.

However, it is preferable that the first silicon oxide particles be the same as the second silicon oxide particles and the second calcium carbonate particles be the same as the first calcium carbonate particles. This is because it is easy to prepare two types of adhesives. The particles being the same means that the particles have the same shape and the same particle size.

The description has been omitted, but in the endoscope 1 in which the emission face 50SA of the illumination unit 50 protrudes from the opening of the distal end face 30SA of the distal end cover 30, the distal end cover 30 and the illumination unit 50 are bonded to each other by the use of the first adhesive 41 containing 10% by mass or more and 60% by mass or less of first calcium carbonate particles, and the step at the boundary between the 30SA of the distal end cover 30 and the emission face 50SA of the illumination unit 50 is covered with the second adhesive 42 containing 10% by mass or more and 60% by mass or less of first silicon oxide particles.

It goes without saying that the first adhesive 41 which fixes the illumination unit 50 to the distal end cover 30 and the second adhesive 42 which eliminates the step between the illumination unit 50 and the distal end cover 30 can be used in the same form and have the same effect as the first adhesive 41 which fixes the image pickup unit 10 to the distal end cover 30 and the second adhesive 42 which eliminates the step between the image pickup unit 10 and the distal end cover 30.

The present invention is not limited to the above described embodiments, modifications and the like, and various changes, combinations and modifications can be made without changing the gist of the present invention.

What is claimed is:

1. An endoscope comprising:
an image pickup unit having an incidence face;
a distal end body including a front face in which an insertion hole is formed, the incidence face of the image pickup unit inserted into the insertion hole protruding from the front face;
a distal end cover that covers a periphery of the insertion hole of the distal end body and has a distal end face including an opening from which the incidence face is protruded;
a first adhesive that bonds the distal end cover to the image pickup unit and contains first calcium carbonate particles; and
a second adhesive that covers a step at a boundary between the distal end face of the distal end cover and the incidence face of the image pickup unit and contains first silicon oxide particles.

2. The endoscope according to claim 1, wherein
the first adhesive contains 0% by mass or more and 30% by mass or less of second silicon oxide particles, and
the second adhesive contains 0% by mass or more and 30% by mass or less of second calcium carbonate particles, wherein
in the first adhesive, a content rate of the silicon oxide particles is smaller than in the second adhesive, and in the second adhesive, a content rate of the calcium carbonate particles is smaller than in the first adhesive.

3. The endoscope according to claim 2, wherein
the second adhesive does not contain the calcium carbonate particles.

4. The endoscope according to claim 1, wherein
in the first adhesive, a thixotropic index is smaller than in the second adhesive.

5. The endoscope according to claim 2, wherein
the first silicon oxide particles and the second silicon oxide particles are spherical and have a same size, and
the first calcium carbonate particles and the second calcium carbonate particles are non-spherical and have a same size.

6. The endoscope according to claim 1, wherein
each of the resin of the first adhesive and the resin of the second adhesive is a curable resi containing a mixture of an epoxy resin and an acrylic rubber.

7. An insertion portion of an endoscope comprising:
an image pickup unit having an incidence face;
a distal end body including a front face in which an insertion hole is formed, the incidence face of the image pickup unit inserted into the insertion hole protruding from the front face;
a distal end cover that covers a periphery of the insertion hole of the distal end body and has a distal end face including an opening from which the incidence face is protruded;
a first adhesive that bonds the distal end cover to the image pickup unit and contains first calcium carbonate particles; and
a second adhesive that covers a step at a boundary between the distal end face of the distal end cover and the incidence face of the image pickup unit and contains first silicon oxide particles.
